(19) United States
(12) Patent Application Publication
(10) Pub. No.: US 2024/0177872 A1
(43) Pub. Date: May 30, 2024

(54) Anxiety Disorders Support System

(71) Applicant: Theresa Jo Whitney, Scranton, PA (US)

(72) Inventor: Theresa Jo Whitney, Scranton, PA (US)

(21) Appl. No.: 18/375,008

(22) Filed: Sep. 29, 2023

Related U.S. Application Data
(63) Continuation of application No. 17/230,996, filed on Apr. 14, 2021, now abandoned.

(51) Int. Cl. G16H 80/00; G16H 10/60; G16H 20/70; G16H 20/10

(52) U.S. Cl. CPC G16H 80/00; G16H 10/60; G16H 20/70; G16H 20/10

(57) ABSTRACT

A method for supporting a person afflicted with an anxiety disorder. The person afflicted with an anxiety disorder is provided with a first electronic communication device such as a cell phone, a smart phone, an iPhone, a laptop computer, or a special purpose custom device in operative communication with at least one trained counselor having access to a second electronic communication device. The counselor may be a member of a team of counselors. When an anxiety attack occurs, the afflicted person requests help via the first electronic communication device from the counselor to recover from the anxiety attack, the request for help including a goal. The afflicted person provides a set of options and chooses one option therefrom. The afflicted person then receives instruction from the counselor to take steps to recover, thus empowering the person afflicted with an anxiety disorder to problem solve for himself or herself.

### FIELD OF THE INVENTION

The present invention relates to supporting anxiety disorders of a person and, more particularly, to a method of supporting the person afflicted with anxiety disorder(s) by consultations with members of a trained support team who guide a person to recovery by providing options and instructions for the afflicted person in real time, empowering the person to problem solve for himself or herself.

### BACKGROUND OF THE INVENTION

Although anxiety is a normal reaction to stress and may even be beneficial in some situations, controlling such anxiety is most often advantageous. People who are afflicted with anxiety disorders experience abnormal feelings of unease, fear, or worry. Anxiety disorders are the most common of mental disorders and affect nearly 30% of adults at some point in their lives. According to the World Health Organization, one in thirteen people suffer from some type of anxiety.

It is estimated that between 2% and 3% of the general population live specifically with panic attacks and that 5% of the population will experience panic in their lives. More than 200,000 U.S. cases of agoraphobia are reported per year. Ages of sufferers range from 14 to senior citizen. This condition is caused by fear. Most people develop this condition after having one or more panic attacks causing excessive worry that another panic attack will occur. The current treatments for panic attacks and agoraphobia recovery include therapies, self-help books on panic attacks, and prescribed medications.

A recent report states that during the Covid-19 pandemic of 2020, anxiety levels increased 62% from the previous year. The American Medical Association reports 46% of women were found to be more likely to say the fear of COVID-19 has negatively impacted their mental health compared with 33% of men. Some 46% of urban and 38% of suburban residents believe COVID-19 has had a negative effect on their mental health compared to 28% of rural area residents. Among adults who have experienced income or job loss due to the pandemic, 46% say the pandemic has negatively affected their mental health too. The most common types of anxiety disorders are defined below.

Generalized anxiety disorder causes someone to feel constantly worried. That person might feel a sense of anxiety while doing daily tasks. Generalized anxiety disorder can be persistent. One might feel very anxious about everyday activities. The person might feel worried about things in life, even when there nothing to be worried about. It can be frustrating and confusing for afflicted people.

Post-traumatic stress disorder (PTSD) is one of the most extreme anxiety disorders. PTSD can occur when a person has experienced particularly traumatic events, such as serving in the military during a war. Victims of various traumatic experiences can develop PTSD due to what they have experienced in their lives. When the traumatic events that an individual experienced come to the forefront of his or her mind, extreme panic can ensue.

Social anxiety disorder can occur when a person has problems interacting with other people. In some cases, a person with extreme social anxiety will not be able to leave the house without feeling an extreme sense of panic.

Obsessive-compulsive disorder (OCD) is characterized by unwanted, repeated thoughts or actions. A person afflicted with OCD might compulsively wash his hands or feel compelled to touch every object in a room before he allows himself to leave it.

Panic disorder results in people experiencing panic episodes that can be extreme at times. Panic attacks result in sudden feelings of terror that strike without warning. These panic episodes can be manifested with physical symptoms and can occur at any time, even during sleep. People experiencing a panic attack may believe they are going crazy. In extreme cases, certain people feel as if they are going to die. Most people experiencing panic attacks exhibit at least one of the following symptoms:
a) sense of terror or impending doom or death;
b) feeling a loss of control;
c) feeling weak, faint, or dizzy;
d) racing heart;
e) chest pains;
f) abdominal pain;
g) breathing difficulties;
h) tingling or numbness in the hands and fingers; and
i) feeling sweaty or having chills.

Although certain medication and surgical implants have been found effective in controlling panic attacks, such a solution may not be practical in real time situations. For example, when a person is in a crowded, public place with no access to medication, his ability to control his mentation can be limited.

What is needed is the ability to communicate with one or more trained members of a support team who can provide options and instructions for the afflicted person in real time, empowering the person to problem solve for himself or herself. It would be advantageous for the 40 million U.S. adults affected by anxiety disorders to have support partners.

### DESCRIPTION OF RELATED ART

U.S. Pat. No. 9,931,500 issued to Lozano, et al. for METHOD OF TREATING DEPRESSION, MOOD DISORDERS AND ANXIETY DISORDERS USING NEUROMODULATION, issued on Apr. 3, 2018, discloses a method and a system for using electrical stimulation and/or chemical stimulation to treat an affective disorder, such as depression. More particularly, the method comprises surgically implanting an electrical stimulation lead and/or catheter that is in communication with a predetermined site which is coupled to a signal generator and/or infusion pump that release either an electrical signal and/or a pharmaceutical resulting in stimulation of the predetermined site thereby treating the mood and/or anxiety.

U.S. Pat. No. 7,410,493 issued to Chen, et al. for METHOD AND DEVICE FOR THE TREATMENT OF ANXIETY DISORDERS, issued on Aug. 12, 2008, discloses a method and device for the treatment of anxiety disorder, including panic attacks. A hammer is used to tap the cutaneous region of the bladder channel on the back until the skin turns pink. The skin on the abdomen, arms, legs, and buttocks can also be used for treatment. The hammer preferably has a magnetic piece in one end of the hammer head. The method can treat anxiety disorder including panic attack, general anxiety, phobia, post-traumatic stress disorder, stress related tension headache, insomnia, and any stress. Patients can use the hammer to treat themselves at home, office, and during traveling.

U.S. Pat. No. 10,898,131 issued to Iocoviello, et al. for SYSTEMS AND METHODS FOR TREATING A PSYCHIATRIC DISORDER, issued on Jan. 26, 2021, discloses systems and methods for treating a subject with a psychiatric disorder in which a therapy session is conducted. In the therapy session, each respective expression image in a plurality of expression images is sequentially displayed and independently associated with an expression. The successive display of images is construed as a tiled series of expression image subsets, each consisting of N expression images. Upon completion of the display of each respective subset, the user is challenged as to whether the first and the last images in the respective subset exhibit the same emotion. A score is determined for the respective subset based on whether the subject learned to respond correctly. The number of images in each subset is adjusted to a new number based on these scores. A treatment regimen is prescribed to the subject for the psychiatric disorder based at least in part on the scores.

U.S. published patent application no. 2011/0027765 on application filed by Nader for METHODS FOR TREATING SOCIAL DISORDERS, published on Feb. 3, 2011, discloses systems and methods for treating patients with an anxiety disorder. The systems comprise a screen for displaying sets of stimuli, a computer to control the display of stimuli onto the screen during at least one treatment session and the ability for the patient to interact with the screen in response to the displayed stimuli. The interaction of the patient with the system during the treatment session is capable of treating patient anxiety associated with an anxiety disorder, such as social anxiety. Also provided are computer programs capable of being used for treating anxiety.

### SUMMARY OF THE INVENTION

In accordance with the present invention, there is provided a method for supporting a person afflicted with an anxiety disorder. A person afflicted with an anxiety disorder is provided with a first electronic communication device such as a cell phone, a smart phone, an iPhone, or a laptop computer compatible with a Google Android-OS or Apple IOS protocol in operative communication with at least one human counselor having access to a second electronic communication device. The counselor may be a member of a team of trained counselors. The first electronic communication device is used to communicate with the counselor via the second electronic communication device when an anxiety attack occurs. The afflicted person requests help via the first electronic communication device from the counselor to recover from the anxiety attack, the request for help including a goal. The afflicted person is encouraged to propose a set of options and to choose one option. The afflicted person then receives a first action goal instruction from the counselor. If the afflicted person declines the action goal, he or she proposes at least one additional option and communicates to the counselor a different goal when the first action goal instruction has been fulfilled. In this way, an anxiety sufferer is empowered to problem solve for himself or herself.

It is therefore an object of the invention to provide a system for helping persons recover from anxiety disorders.

It is a further object of the present invention to provide a system for empowering persons subjected to anxiety disorders to problem solve for themselves.

It is another object of the present invention to provide a system for training members of a support team to help such afflicted people in real time.

It is a further object of the present invention to provide a system for collecting and analyzing data by using a behavior-based application, allowing for shared growth and progress to team members and to be shared with the medical community for educational purposes, in helping to create a health culture.

These and other objects and advantages of the present invention are more readily apparent with reference to the following detailed description and the accompanying drawings.

### BRIEF DESCRIPTION OF THE DRAWINGS

A complete understanding of the present invention may be obtained by reference to the accompanying drawings, when considered in conjunction with the subsequent detailed description, in which:

FIG. 1 is a schematic view of an afflicted person's communication device having a mobile chat-based app installed thereon in an initial state in accordance with the present invention;

FIG. 2 is a schematic view of support team members' communication device after a panic button has been pressed;

FIG. 3 is a schematic view of an afflicted person's communication device;

FIG. 4 is a schematic view of support team members' communication devices;

FIG. 5 is another schematic view of an afflicted person's communication device;

FIG. 6 is a graph representing collected data;

FIG. 7 is a general flow chart of operations; and

FIG. 8 is a flow chart of operations more specific than that shown in FIG. 7.

Like reference numerals refer to like parts throughout the several views of the drawings.

### DETAILED DESCRIPTION OF THE PREFERRED EMBODIMENT

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

A method of supporting a person with anxiety disorders is provided by facilitating online, real time consultations with a support team that guides a person to recovery by encouraging options and providing instructions for the afflicted person in real time, empowering the person to problem solve for himself or herself. The person afflicted with an anxiety disorder is provided with a first electronic communication device in operative communication with at least one counselor having access to a second electronic communication device. The counselor may be a member of a team of trained counselors. The first electronic communication device is used to communicate with the counselor via the second electronic communication device when an anxiety attack occurs. The afflicted person requests help from the counselor to recover from the anxiety attack. The afflicted person is encouraged to propose a set of options and to choose one option. The afflicted person then receives a first action goal instruction from the counselor to recover.

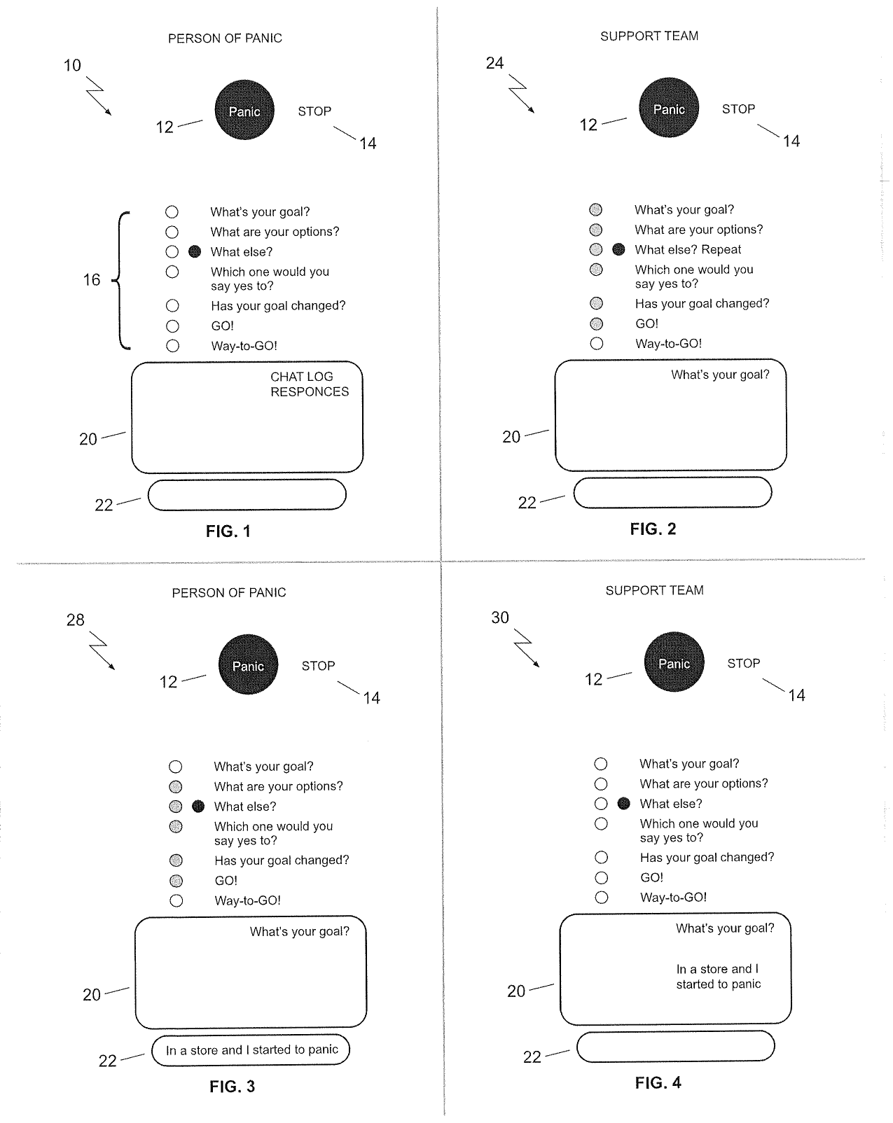

Referring now to FIG. 1, there is shown a schematic view of a display 10 of an afflicted person's communication device (not shown) having a mobile chat-based app installed thereon in an initial state. The communication device in the preferred embodiment is a Google Android-OS or an Apple iOS chat-based application, but other brands of cell phone, a smart phone, iPhone, or a laptop computer can be used as long as they are compatible with Android-OS or iOS protocols. Alternatively, a custom, special purpose communication device with or without touch screen capability can be used.

A display 10 of the communication device includes a Panic button 12 and a STOP button 14. STOP button 14 is a safety button that can be pressed by a team member or counselor for inappropriate responses in accordance with the inventive process. Team members may be professional health workers or may be lay people trained to respond to panic messages communicated by afflicted persons. A menu 16 of six steps of the inventive decision-making process is also displayed in accordance with the invention, each step being highlighted by a counselor as appropriate. In the preferred embodiment, the six steps are:
1) What's your goal?
2) What are your options?
3) What else?
4) Which one would you say yes to?
5) Has your goal changed?
6) GO!

It should be understood that the precise language of the foregoing questions can be modified without departing from the scope of the invention, as long as the essence and meaning of such questions are preserved.

Below the six-step menu 16 are displayed two chat boxes 20, 22. The first chat box 20 displays messages sent by the afflicted person and the first responder team member, and the second chat box 22 displays the afflicted person's messages as they are typed.

Referring now to FIG. 2, there is shown a schematic view of a display 24 of support team members' communication devices after Panic button 12 has been pressed by an afflicted person. The first team member who responds (i.e., the first responder) clicks on the menu item, “What's your goal?” All members of the support team receive a message requesting them to open their respective mobile apps.

Referring now to FIG. 3, there is shown a schematic view of a display 28 of an afflicted person's communication device when he or she types a message in the second chat box 22 to the first responder team member. The message that the afflicted person types a description of his or her action goal (e.g., “In a store and I started to panic”).

Referring now to FIG. 4, there is shown a schematic view of a display 30 of support team members' communication devices indicating that a first responder is engaged with the afflicted person and placing all other members in standby mode. The standby mode disables support team members from participating in the session, but each member can watch the session transpire between the afflicted person and the first responder, as shown in first chat box 20.

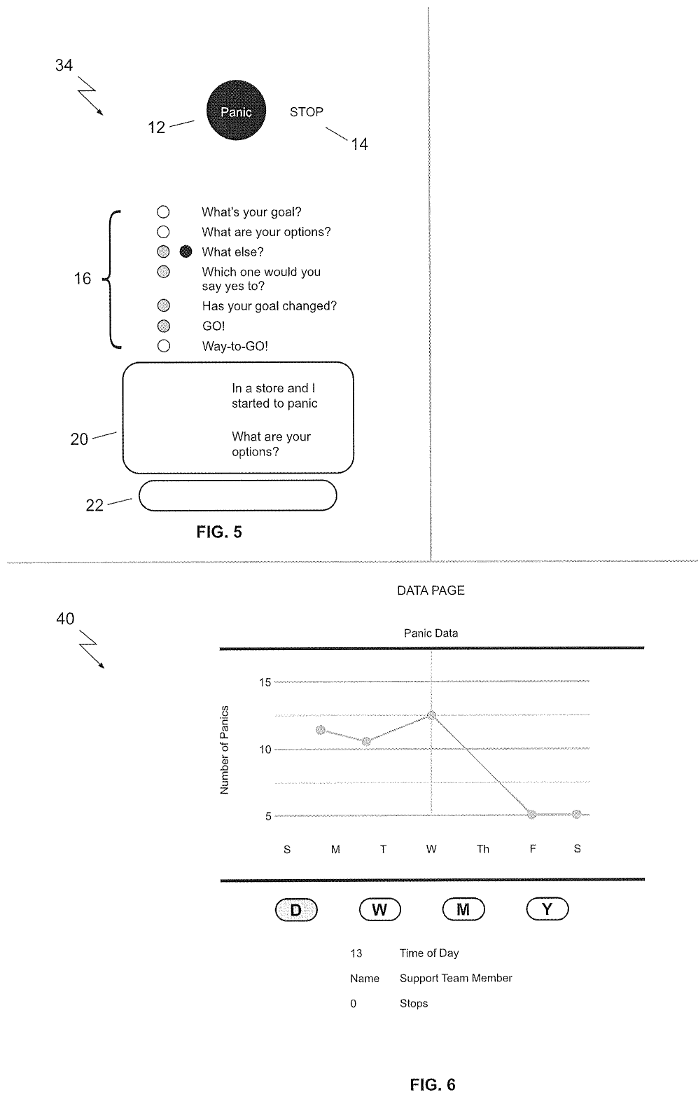

Referring now to FIG. 5, there is shown a schematic view of a display 34 of remaining support team members' communication devices in standby mode. The afflicted person, after typing his or her action goal, then receives the message, “What are your options?” in the first chat box 20 from the first responder. The afflicted person can then list all options, both realistic and unrealistic, to proceed in the present situation.

Once the first responder receives the afflicted person's options, the first responder sends the next question, “What else?” to the afflicted person. The “What else?” menu item is highlighted. This response continues and extends the afflicted person's list of options. The more options that are listed by the afflicted person, the less threatening the attack becomes. This, in turn, lowers anxiety by having the afflicted person recognize choices.

The first support responder can repeat the question, “What else?” until the afflicted person has generated and typed all options. In response to the question, “Which one would you say yes to?”, the afflicted person chooses an option, at which point the first support responder highlights the icon, “Has your goal changed?” If the goal has changed, the system loops back to the beginning of the menu 16, requesting the afflicted person state a new goal, and so on. Once the afflicted person has chosen an option and answered the first support responder's most recent question in the negative, the first support responder highlights the GO! icon on the afflicted person's display. The afflicted person is then free to implement the chosen option. The process results in the afflicted person being empowered to problem solve for himself or herself and to take the necessary step(s) for recovery.

After the afflicted person types the last option of the last goal and presumably executed the chosen option, the celebration of success (“Way-to-GO!”) icon is highlighted on the afflicted person's communication device under direction of the first support responder.

Referring now to FIG. 6, there is shown a graph 40 of collected data, the number of panic attacks captioned along the Y-axis and the days of the week captioned along the X-axis. The invention analyzes collected data including, but not limited to the time of day, the number of successful sessions, the name of the support team member, the number of stops, etc. by using a behavior-based application. These analytics allow for shared growth and progress to team members and to be shared with the medical community for educational purposes, in helping to create a health culture. This inventive mobile app also contains a built-in matrix that can gauge panic attack occurrences by the day, week, month, and/or year. The system offers shared information on the time of day the panic is occurring, how many successful sessions, which support team members coached each session, and how many stops were engaged. All of these findings can be useful in future research studies of panic disorders and recovery support teams.

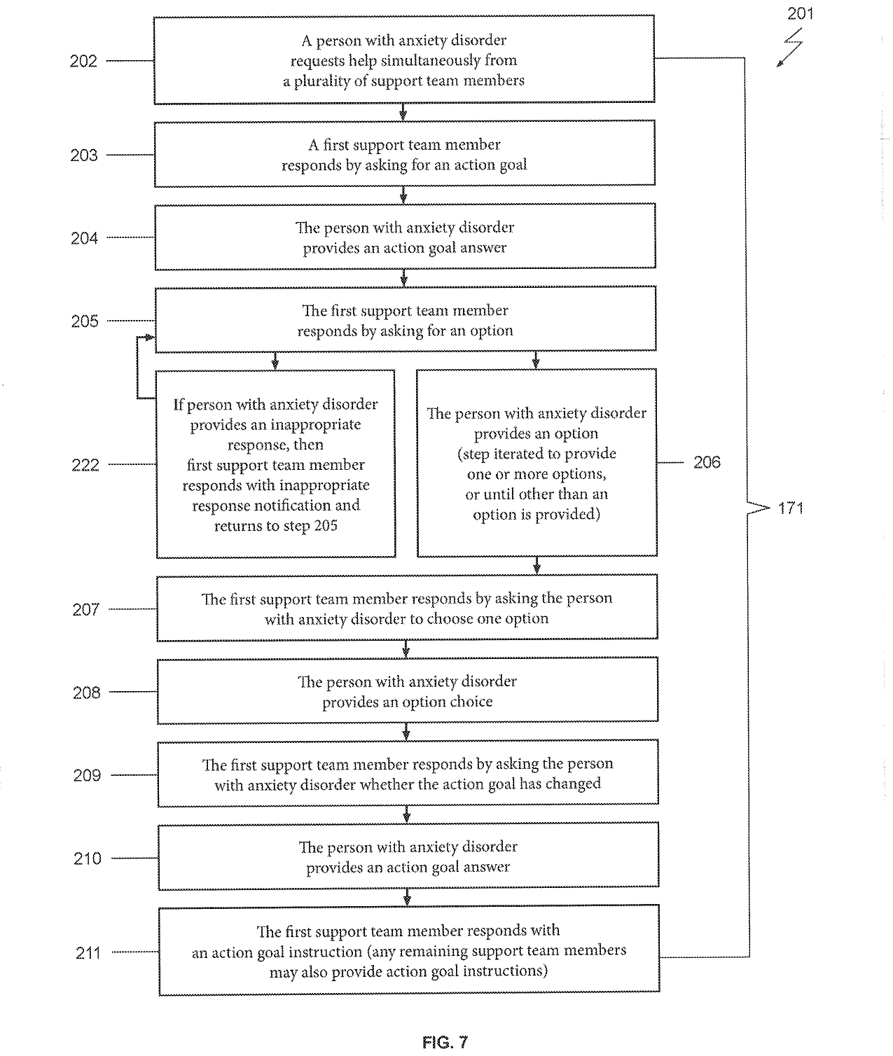

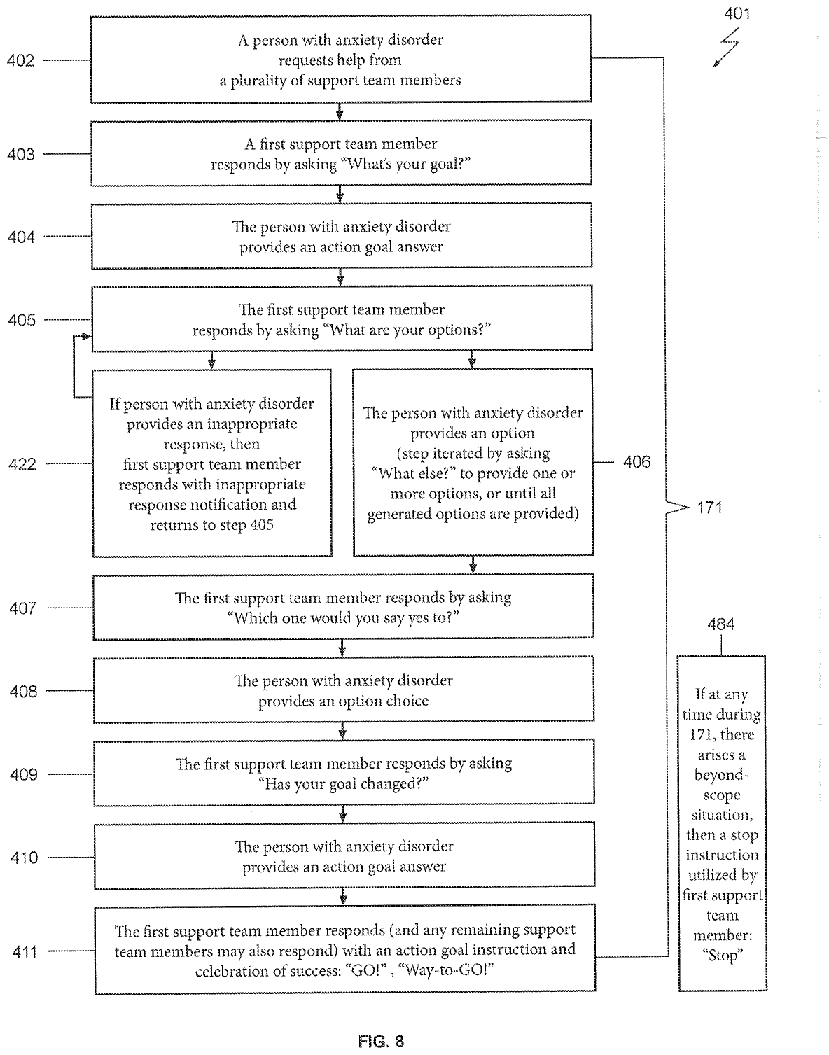

Referring now to FIG. 7, there is shown a general flow chart of system operations identified by reference numeral 201. A person with an anxiety disorder requests help from a plurality of support team members, step 202. The support team member who first responds is known as the “first responder.” That first support team member responds to the request for help by asking for an action goal, step 203, using only pre-selected language to communicate with the afflicted person when responding, asking, providing, instructing, and the like.

The afflicted person types an action goal answer, step 204. The first support team member then asks the afflicted person for at least one option, step 205. Once the afflicted person types an option, step 206, the first support team member can ask for additional options, which the afflicted person can provide by typing, one at a time, step 206. The first and any subsequent options are displayed to all parties. If the afflicted person then provides an inappropriate response, step 222, the first support team member responds by pressing the STOP button 14 (FIG. 1) to indicate the response was inappropriate. That notification is followed by a request by the first responder for an option, step 205.

Once all options have been generated and stated by the afflicted person, step 206, the first support team member requests the afflicted person to choose one option, step 207, which the afflicted person then chooses, step 208.

At this point, the first support team member asks the afflicted person whether the action goal has changed, step 209, to which the afflicted person responds, step 210. The first support team member then communicates an action goal instruction to the afflicted person, step 211.

While communications, steps 202-211 (indicated by reference numeral 171), occur between the first support team member and the afflicted person, other support team members are in standby mode, wherein such support team members can observe the ongoing communications and can communicate with one another by responding to the action goal answer. Once the support team members receive a session finished alert, any of such members can respond to the afflicted person's action goal answer with one or more additional action goal instructions.

Referring now to FIG. 8, there is shown a flow chart of system operations 401 more specific than that described with reference to FIG. 7. A person with an anxiety disorder requests help from a plurality of support team members, step 402. The first support team member responds to the request for help by asking “What's your goal?” step 403, using only pre-selected language to communicate with the afflicted person when responding, asking, providing, instructing, and the like.

The afflicted person types an action goal answer, step 404. The first support team member then asks the afflicted person for at least one option with the question, “What are your options?” step 405. Once the afflicted person types an option, step 406, the first support team member can ask for additional options with the question “What else?” which the afflicted person can provide by typing, one at a time, step 406. The first and any subsequent options are displayed to all parties. If the afflicted person then provides an inappropriate response, step 422, the first support team member responds with an inappropriate response notification followed by another request for an option, step 405.

Once all options have been generated and stated by the afflicted person, step 406, the first support team member requests the afflicted person to choose one option with the question, “Which one would you say yes to?” step 407, which the afflicted person then chooses, step 408.

At this point, the first support team member asks the afflicted person whether the action goal has changed by asking the question, “Has your goal changed?” step 409, to which the afflicted person responds, step 410. The first support team member then communicates an action goal instruction to the afflicted person, step 411, with the statement, “GO!” and, after completion of the action, “way to GO!” as a celebration of success.

While communications, steps 402-411 (indicated by reference numeral 171), occur between the first support team member and the afflicted person, other support team members are in standby mode, wherein such support team members can observe the ongoing communications and can communicate with one another by responding to the action goal answer. Once the support team members receive a session finished alert, any of such members can respond to the afflicted person's action goal answer with one or more additional action goal instructions.

If, at any time during communications 171, a response is generated that is beyond the scope of the situation, a STOP instruction is sent by the first support team member to the afflicted person, step 484.

It should be understood that the dialog questions and answers presented above are merely illustrative and the invention is intended to cover all manner of statements that result in messages having similar meanings.

All references throughout this application, for example patent documents including: issued or granted patents or equivalents; patent application publications; and non-patent literature documents; or other source material are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that, although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a great number of variations of the devices, device components, and method steps set forth in the present description. As will be obvious to one of skill in the art, methods and devices useful for the present methods can include a great number of optional composition and processing elements and steps.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when mobile processors or apps are claimed, it should be understood that processors and apps known and available in the art prior to Applicant's invention are meant to be included in such claims.

As used herein, “comprising” is synonymous with “including,” “containing,” or “characterized by,” and is inclusive or open-ended and does not exclude additional, unrecited elements or method steps. As used herein, “consisting of” excludes any element or step not specified in the claim element. As used herein, “consisting essentially of” does not exclude materials or steps that do not materially affect the basic and novel characteristics of the claim. In each instance herein any of the terms “comprising”, “consisting essentially of,” and “consisting of” may be replaced with either of the other two terms. The invention illustratively described herein suitably may be practiced in the absence of any element or elements, step or steps, limitation or limitations which is not specifically disclosed herein.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

What is claimed is:

1. A method for supporting a person afflicted with an anxiety disorder in real time, the steps comprising:
a) providing a person afflicted with an anxiety disorder with a first electronic communication device in operative communication with at least one trained counselor having access to a second electronic communication device;
b) using the first electronic communication device to communicate with the at least one counselor via the second electronic communication device when the person afflicted with an anxiety disorder uses the first electronic communication device to request help from the at least one counselor when an anxiety attack occurs;
c) requesting help via the first electronic communication device from the at least one counselor for guidance therefrom to recover from the anxiety attack, the request for help including a goal;
d) generating and communicating a set of at least one option via the first electronic communication device to the at least one counselor;
e) choosing one option via the first electronic communication device from the set of at least one option; and
f) receiving a first action goal instruction via the first electronic communication device from the at least one counselor.

2. The method for supporting a person afflicted with an anxiety disorder in real time in accordance with claim 1, the steps further comprising:
g) providing at least one additional option via the first electronic communication device to the at least one counselor when the action goal instruction of step (f) is declined by the person afflicted with an anxiety disorder.

3. The method for supporting a person afflicted with an anxiety disorder in real time in accordance with claim 1, the steps further comprising:
g) providing the counselor with a different goal when the first action goal instruction has been fulfilled by the person afflicted with an anxiety disorder.

4. The method for supporting a person afflicted with an anxiety disorder in real time in accordance with claim 1, wherein the first and second electronic communication devices are chosen from a set of devices consisting of: a cell phone, a smart phone, an iPhone, a laptop computer, and a special purpose custom device.

5. The method for supporting a person afflicted with an anxiety disorder in real time in accordance with claim 1, the steps further comprising:
g) using a behavior-based application to collect and analyze data comprising information generated by the person afflicted with an anxiety disorder in steps (c) and (e).

6. A method for supporting a person afflicted with an anxiety disorder in real time, the steps comprising:
a) providing a person afflicted with an anxiety disorder with a first electronic communication device in operative communication with at least one trained counselor being a member of a team thereof and having access to a second electronic communication device;
b) using the second electronic communication device to receive communications from the person afflicted with an anxiety disorder via the first electronic communication device when an anxiety attack occurs;
c) receiving a help request via the second electronic communication device from the person afflicted with an anxiety disorder to recover from the anxiety attack, the request for help including a goal;
d) requesting a set of at least one option via the second electronic communication device from the person afflicted with an anxiety disorder;
e) receiving a preferred option via the second electronic communication device from the set of at least one option; and
f) issuing a first action goal instruction via the second electronic communication device to the person afflicted with an anxiety disorder.

7. The method for supporting a person afflicted with an anxiety disorder in real time in accordance with claim 6, wherein steps (b)-(f) are performed by at least one other member of the team of counselors.

8. The method for supporting a person afflicted with an anxiety disorder in real time in accordance with claim 7, the steps further comprising:
g) receiving at least one additional option via the second electronic communication device from the person afflicted with an anxiety disorder when the action goal instruction of step (f) is declined thereby.

9. The method for supporting a person afflicted with an anxiety disorder in real time in accordance with claim 6, the steps further comprising:
g) receiving a different goal by at least one other member of the team of counselors when the first action goal instruction has been fulfilled by the person afflicted with an anxiety disorder.

10. The method for supporting a person afflicted with an anxiety disorder in real time in accordance with claim 6, wherein the first and second electronic communication devices are chosen from a set of devices consisting of: a cell phone, a smart phone, an iPhone, a laptop computer, and a special purpose custom device.

11. The method for supporting a person afflicted with an anxiety disorder in real time in accordance with claim 6, the steps further comprising:
g) providing a behavior-based application for collecting data comprising information generated by the person afflicted with an anxiety disorder in steps (c) and (e) in the course of guiding persons afflicted with anxiety disorders to recovery.

12. The method for supporting a person afflicted with an anxiety disorder in real time in accordance with claim 11, the steps further comprising:
h) using the behavior-based application to analyze the collected data.

13. The method for supporting a person afflicted with an anxiety disorder in real time in accordance with claim 7, wherein any member of the team of trained counselors provides action goal instructions to the person with an anxiety disorder.

14. The method for supporting a person afflicted with an anxiety disorder in real time in accordance with claim 6, the steps further comprising:
g) after requesting an option choice from the person afflicted with an anxiety disorder, step (d), asking the person afflicted therewith whether the first action goal has changed.

15. The method for supporting a person afflicted with an anxiety disorder in real time in accordance with claim 14, the steps further comprising:
h) after receiving an affirmative answer from the person afflicted with an anxiety disorder to the question whether the action goal has changed, step (f), and requesting a second action goal therefrom.

16. The method for supporting a person afflicted with an anxiety disorder in real time in accordance with claim 7, wherein any member of the team of trained counselors provides subsequent action goal instructions to the person afflicted with an anxiety disorder.

17. The method for supporting a person afflicted with an anxiety disorder in real time in accordance with claim 7, wherein all responses and instructions generated by members of the team of counselors are pre-selected.

18. A method for supporting a person afflicted with an anxiety disorder in real time, the steps comprising:
a) communicating with a person afflicted with an anxiety disorder via communication devices provided to the person afflicted with an anxiety disorder and to a trained counselor; and
b) responding to requests of, and guiding, the person afflicted with an anxiety disorder to take action to recover therefrom.

19. The method for supporting a person afflicted with an anxiety disorder in real time in accordance with claim 18, the steps further comprising:
c) encouraging the person afflicted with an anxiety disorder to propose a goal, to propose a set of options, to select one of the set of options, to execute the selected option, and to congratulate the person afflicted with an anxiety disorder once the selected option has been successfully executed thereby; and
d) whereby the person afflicted with an anxiety disorder is empowered to problem solve.

20. The method for supporting a person afflicted with an anxiety disorder in real time in accordance with claim 19, the steps further comprising:
e) providing a behavior-based application for collecting and analyzing data comprising information generated by the person afflicted with an anxiety disorder in step (c) in the course of guiding the person afflicted with anxiety disorders to recovery.
